(19) United States
(12) Patent Application Publication
(10) Pub. No.: US 2024/0404689 A1
(43) Pub. Date: Dec. 5, 2024

(54) TELEMEDICINE SYSTEM AND METHOD FOR MONITORING AND CONTROLLING PARAMETERS OF ARTIFICIAL LUNG VENTILATION DEVICE

(71) Applicant: UAB "TELTONIKA IOT GROUP", Vilnius (LT)

(72) Inventors: Povilas Andrijauskas, Vilnius (LT); Tomas Motuzas, Vilnius (LT); Martynas Osauskas, Vilnius (LT); Juozas Sadauskas, Vilnius (LT); Giedrius Zukauskas, Vilnius (LT); Tomas Zamaras, Vilnius (LT); Audrius Andrijauskas, Vilnius (LT); Tomas Jovaisa, Vilnius (LT)

(21) Appl. No.: 18/325,243

(22) Filed: May 30, 2023

(51) Int. Cl. G16H 40/40; A61M 16/00; G16H 10/60; G16H 40/67; G16H 80/00

(52) U.S. Cl. CPC G16H 40/40; A61M 16/0051; G16H 10/60; G16H 40/67; G16H 80/00

(57) ABSTRACT

Described is an artificial lung ventilation device (ALV) parameter monitoring and control system including more than one ALV, a server, a remote ALV parameter monitoring and control device, and a two-way voice communication module. Each ALV has a patient identification device. The server has a layout scheme of the ALVs in the premises. The telemedicine system identifies each patient and ALV. Ventilation modes, ALV parameters, alarms, available ventilation mode and at least one ALV parameter can be monitored and changed remotely. The server with ALV and ALV parameter monitoring and control device can be connected via physical connections, wireless communication or mobile Internet. The ALV has an integrated or stand-alone IoT unit—in this case the communication in the system can be easily changed, e.g. from 4G to 5G. Also described is a telemedicine method for monitoring and controlling ventilation modes, alarms and parameters of more than one ALV.

### FIELD OF THE INVENTION

The invention belongs to the area of medical devices, and specifically to the remote control of artificial lung ventilation devices by telemedicine solutions.

### BACKGROUND OF THE INVENTION

An artificial lung ventilation device (ALV) is a device that supplies a breathing gas mixture to the patient's airways to ensure oxygenation and removal of CO2 from the body. ALV is used in various clinical conditions, when acute or chronic respiratory failure occurs: lung diseases, head or spine injuries, stroke, sudden cardiac arrest, severe acute respiratory syndrome, pneumonia and others. During the COVID-19 pandemic, there has been a significant increase in the need for ALV, as 5% of patients with COVID-19 develop acute respiratory failure and require artificial lung ventilation. Lung ventilation is divided into invasive and non-invasive. Applied ventilation modes can be compulsory and auxiliary depending on the patient's condition and respiratory function. The delivery of the breathing gas mixture to the patient's airways can be volume controlled, pressure controlled or mixed. In intensive care units, mechanical ventilators with controlled ventilation are used, which take over the control of the patient's breathing in full or partially control the patient's breathing. A tube is inserted into the patient's trachea, through which a mixture of breathing gases is supplied to the airways and carbon dioxide is removed. The disease caused by the COVID-19 virus manifests itself with a particularly prominent level of contagiousness through airborne droplets. This leads to extreme working conditions for the medical staff who work directly with patients suffering from this disease. Patients with a contagious disease are usually treated in isolated areas, which are separated from premises not infected with the COVID-19 virus. In the case of COVID-19 or other contagious diseases, medical personnel wear protective equipment in infected areas, such as: suits, goggles, respirators, gloves, gowns, etc. It takes 5-10 minutes to put this equipment on. The treatment of patients on artificial lung ventilation requires special attention. This group of patients is characterized by multiple organ failure and a very dynamic course of the condition, often requiring quick decisions and changing the settings of devices that ensure vital functions. A delay in changing the modes and parameters of the ALV device can cause damage to the patient's health. While in a “clean” area, the doctor not only cannot see the ALV parameters of a remote patient, but also to make changes, he has to get to the patient, losing a lot of time due to putting on protective equipment, etc. When human resources allow having a doctor near all patients 24/7, this problem is not so high, excluding the fact that an extended stay in an infected area increases the probability of the doctor becoming infected. However, with the lack of specialized doctors who are able to correctly operate ALV devices, this becomes a highly burdensome circumstance.

One of the solutions proposed in the world to solve the mentioned problems is telemedicine, which provides remote solutions for monitoring patients or controlling medical devices, thus reducing or completely avoiding contacts between the patient and the staff of the medical institution and shortening the time for performing an active medical action. Remote monitoring and control of ALV would reduce the chance of infection and affect the patient's health or even survival.

Patent application EP3624131A1 (published on 18 Mar. 2020) describes a solution that enables remote monitoring of ALV parameters. However, there is no possibility to change the parameters of ALV remotely, so, although less often, the staff of the medical institution still has to go to the patient. In addition, there is no possibility to combine many ALVs into a single system, so if there are many patients and ALVs, many described ALV monitoring devices will be required, which is space-consuming, inconvenient and expensive.

Patent application US20180082033A1 (published on 22 Mar. 2018) describes a device that remotely monitors and can automatically change some ALV parameters. However, it provides an automatic control of only a few essential ALV parameters. The staff of the treatment facility cannot control the selected ALV parameters remotely, they still have to get to the patient. In addition, each ALV has a stand-alone monitoring and control apparatus, so it makes inconvenient and expensive to monitor many patients.

Patent application US20020120676A1 (published on 4 Jan. 2005) describes an ALV monitoring system for combining more than one ALV into a common system. This system has disadvantages because the system is only for monitoring the ALV parameters, there is no possibility to control the ALV parameters remotely.

### SUMMARY OF THE INVENTION

Described herein is a remote monitoring and control system for artificial lung ventilation device (ALV) parameters, ventilation modes and alarm signals, including more than one ALV, a server, a remote ALV parameter monitoring and control device, and a voice communication module. Each ALV has a patient identification device, and the server has a diagram of the placement of ALVs in the premises, so this telemedicine system identifies each ALV and allows monitoring and changing the parameters of more than one ALV remotely, which reduces the possibility of contamination and saves time when visiting patients. The server with ALV and ALV parameter monitoring and control device can be connected via physical connection, wireless communication (wi-fi, Bluetooth) or mobile internet (2G/3G/4G or 5G). The ALV has a stand-alone Internet of Things unit, so the communication between the server and the ALV parameter monitoring and control device can be easily changed, e.g. from 4G to 5G. The communication is encrypted, and the communication requires authorization and identification, so outsiders cannot access the system and control the ALV parameters. The ALV parameter monitoring and control system can monitor ventilation modes and the following ALV parameters: oxygen concentration (FiO2), respiratory rate (RR), inspiratory time (Tinsp), expiratory time (Texp), inspiratory pressure (Pinsp), inspiratory-expiratory time ratio (I:E ratio), inspiratory single volume (VTinsp), maximum pressure (Pmax), positive end-expiratory pressure (PEEP), determination of spontaneous breathing sensitivity (Spont trigger), backup mode selection (Backup mode), pressure maintenance (Pressure support), total ventilation time, inspiratory minute volume (MVinsp), expiratory single volume (VTexp), expiratory minute volume (MVexp), respiratory gas leakage (Air leak), total respiratory frequency (Rtot), respiratory gas flow (Flow), lung tissue compliance (Compliance), peak pressure (Ppeak), pressure plateau index (Pplato), oxygen saturation (SpO2) and end-expiratory carbon dioxide (ETCO2) as well as ALV calculated outputs, backup power battery charge level, date and time and alarm signals. The system can change ventilation modes and at least one of these ALV parameters. Also described is a telemedicine method for monitoring and controlling parameters of more than one ALV, which allows combining multiple ALVs into a common system and remotely monitoring and changing selected ALV parameters.

Embodiments described herein are intended to address problems and disadvantages of the prior art. The described ALV system has one or more of the following advantages: more than one ALV can be connected into a common system; there is two-way voice communication between ALV and ALV parameters monitoring and control system; each ALV has a patient identification device that allows easy identification of each patient and ALV; the monitoring and control device has two modules: the ALV parameters monitoring module and the ALV parameters monitoring and control module; integrated alarm system adapted for remote monitoring of ALV parameters.

### BRIEF DESCRIPTION OF THE DRAWINGS

Embodiments of the invention will now be described with reference to the appended drawings in which:

FIG. 1 is a block diagram of the artificial lung ventilation device parameter monitoring and control system.

### DETAILED DESCRIPTION

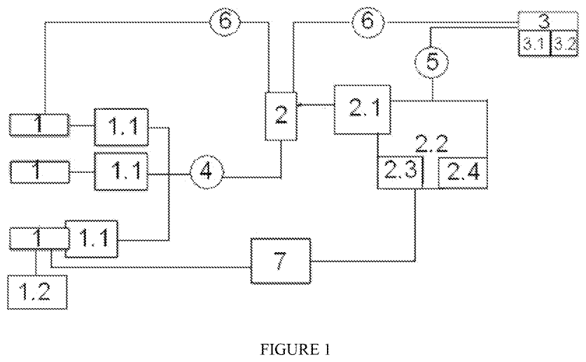

Disclosed herein is a telemedicine system for monitoring and controlling the parameters of an artificial lung ventilation device (ALV) (1) and a method for combining more than one ALV (1) into a common telemedicine system and remotely monitoring and changing the parameters of the ALV (1). FIG. 1 is a block diagram of the ALV (1) parameter monitoring and control system. In FIG. 1 and as explained further below, 1—ALV, 1.1—IoT unit, 1.2—data input and output device, 1.3—patient identification device, 1.4—backup power battery, 1.5—Internet service, 2—server, 2.1—virtual machine, 2.2—telemedicine software, 2.3—database, 2.4—layout scheme of ALVs in premises, 3—device for monitoring and controlling ALV parameters, 3.1—ALV parameter monitoring module, 3.2—ALV parameter monitoring and control module, 4—communication between ALV and server, 5—communication between server and ALV parameter monitoring and control device, 6—two-way voice communication module, 7—alarm system.

The system includes:
ALV (1);
server (2);
ALV parameter monitoring and control device (3);
two-way voice communication (6).

ALV (1) is a medical device designed to artificially ventilate the lungs of patients. During ventilation, a mask or tube is used, through which breathing gases are supplied to the patient's lungs: air, oxygen or a mixture of oxygen with other gases. The concentration of the supplied breathing gas mixture is determined by the FiO2 parameter, which is controlled in the ALV device. Artificial lung ventilation can be controlled in either volume or pressure modes. Mechanical ALV (1) is mostly used. ALV (1) includes IoT unit (1.1), data input and output device (1.2), patient identification device (1.3), backup power battery (1.4) and accessories: non-invasive ventilation face mask or intubation tube, exhalation filter, breathing filter, patient circuit, exhalation valve, breathing gas supply line, ALV power cable and other standard accessories, thanks to which breathing gas of the required composition, pressure and volume is supplied to the patient's lungs. ALV (1) can be volume-controlled when the volume of air delivered to the lungs is controlled; pressure-controlled, where the pressure of the air supplied to the lungs is controlled; or mixed, controlled by volume and pressure. The ventilation modes required for ALV (1) can be selected and can be changed during intensive care. ALV (1) has physical connections to external devices. Such connections can be USB, HDMI, DVI, alarm outputs for connecting the device to an existing alarm system, and others.

In one implementation option of the invention, the ALV (1) operates when the ALV (1) parameter monitoring and control system has a standard breathing gas pressure.

In another implementation option of the invention. ALV (1) operates when the operating pressure of the central breathing gas supply system in the ALV (1) parameter monitoring and control system is lower than required by the standard. This is important in the event of a pandemic or other disaster, when a large number of ALV (1) are connected to the ALV (1) parameter monitoring and control system, and there is not enough gas supply to maintain high pressure. For example, when the standard pressure is 2.8 bar, the ALV (1) can also operate at a pressure of 2 bar. This ensures reliable operation of the ALV (1) in case of an overloaded central breathing gas supply system.

The (IoT) Internet of Things unit (1.1) is a device thanks to which the ALV (1) maintains communication with the server (2). More than one ALV (1) can be connected to the ALV parameter monitoring and control system (3) (FIG. 1). In one implementation option of the described invention, the ALV (1) parameter monitoring and control system includes 20 ALV (1) or more. There are two feasible options for connecting the IoT unit (1.1) to the ALV (1). In the first case, the IoT unit (1.1) is integrated into the ALV (1). In this case. ALV (1) is compact, convenient to use, does not require wiring. Otherwise, the ALV (1) has a replaceable IoT unit (1.1) which is available as a stand-alone attachment. In this case, the advantage is that when communication transmission technologies change, it is possible to easily replace one communication with another without changing the ALV itself (1). When replacing the IoT unit (1.1) the Internet communication can easily be changed. e.g. from 4G to 5G.

The data input and output device (1.2) is one or more devices, thanks to which it is possible to monitor and change the parameters of the ALV (1), the ventilation modes used, and to monitor the alarm signals in the presence of direct proximity to the ALV (1) and the patient. The data input and output device (1.2) can be a computer, tablet, mobile phone or other device that has a screen for monitoring the parameters of the ALV (1) and a multifunction knob, keyboard, mouse or touch screen for changing the parameters of the ALV (1). The data input and output device (1.2) has a microphone, speaker, video camera or other means necessary for the transmission of voice and video data. Thanks to the data input and output device (1.2), it is possible to set parameters of the ALV (1) mode, alarm limits and enter patient data.

In one implementation option, the patient identification device (1.3) determines the location of each ALV (1) with the help of wi-fi technology, which allows monitoring and positioning of the ALV (1) location. In another implementation option, when a wired connection is used, the location of the ALV (1) is determined by the routing table, gateway numbers, and other addresses.

The ALV (1) also has a backup power battery (1.4) that can ensure the functionality of the ALV (1) without an external power source.

In a separate case, the ALV (1) additionally has an Internet service (1.5), which allows the ALV (1) to be connected directly to the ALV parameter monitoring and control device (3). In this case, the ALV (1) parameter monitoring and control system includes the ALV (1) and the ALV parameter monitoring and control device (3), and the server (2) is not included in the system. When the ALV (1) has an Internet service (1.5), accessing the Internet service (1.5) remotely, the ALV (1) web page with the ALV (1) parameters monitoring and control is opened in the browser window of the ALV parameter monitoring and control device (3) functionality. This is a convenient and cheap way to manage ALV (1) remotely, when the medical institution acquires only one or several ALVs (1), since in this case there is no need to purchase a server (2).

As the patient's condition changes, it is necessary to monitor and change ventilation parameters and/or ventilation modes accordingly. In the case of the described invention, the following ALV (1) parameters are monitored, which can be changed directly by ALV (1) or remotely: oxygen concentration (FiO2), respiratory rate (RR), inspiratory time (Tinsp), inspiratory pressure (Pinsp), inspiratory-expiratory time ratio (I:E ratio), inspiratory single volume (VTinsp), maximum pressure (Pmax), positive end-expiratory pressure (PEEP), determination of spontaneous breathing sensitivity (Spont trigger), selection of backup mode (Backup mode) and pressure maintenance (Pressure support). ALV (1) also has the following parameters, the values of which cannot be changed, but which are displayed during artificial lung ventilation: total ventilation time, inspiratory minute volume (MVinsp), expiratory single volume (VTexp), expiratory minute volume (MVexp), expiratory minute volume (MVexp), gas leakage (Air leak), total breathing frequency (Rtot) (equal spontaneous frequency+mechanical breathing frequency), flow of breathing gas (Flow), continuity of lung tissue (Compliance), peak pressure (Ppeak), pressure plateau index (Pplato), oxygen saturation/pulse oximetry (SpO2), end-expiratory carbon dioxide (ETCO2), respiratory gas flow, pressure-volume curves, pulse oximetry and CO2 curves.

The ALV (1) parameter monitoring and control system has a server (2) which is connected to the ALV (1) and the ALV parameter monitoring and control device (3), so that the server (2) receives data from more than one ALV (1) and forwards the data to the ALV parameter monitoring and control device (3). The server (2) can be stationary, located e.g. in the premises of a treatment facility or service provider, or a virtual one located e.g. in the virtual cloud.

In the described case, the ALV (1) parameter monitoring and control system has a physical server (2) containing a virtual machine (2.1) with telemedicine software (2.2), a database (2.3) and a layout scheme of ALVs in the premises (2.4) (FIG. 1). The database (2.3) stores data about patients. ALV (1), different parameters and parameter settings of ALV (1), accessories used in ALV (1), alarm history and other related data. The telemedicine software (2.2) controls the reception and forwarding of data to/from the ALV (1) and the ALV parameter monitoring and control device (3), and the recording and retrieval of data from the database (2.3). The layout scheme of ALVs in the premises (2.4) collects data on the location of each ALV (1) in the treatment facility and provides a general diagram that shows the layout of all ALVs (1) in the system. The layout scheme of ALVs in the premises (2.4) can be of several levels-a diagram showing resuscitation stations, a drawing of a ward of resuscitation stations or interactive icons of ALV. It is possible to choose that the layout scheme of ALVs in the premises (2.4) shows several desired ALV (1) parameters or alarms next to each ALV (1). For example, critical parameters, their values and alarms can be displayed. The layout scheme of ALV in premises (2.4) is interactive: after pressing the icon of a specific ALV (1), other parameters and other related information of this specific ALV (1) appear on the screen of the ALV parameter monitoring and control device (3).

In another implementation option of the described invention, a physical server (2) may not be present, instead a virtual server (2) may be present. In one implementation option, a server (2) located in a private virtual cloud is used.

In another implementation option, a server (2) located in the virtual cloud of the service provider is used.

The ALV (1) parameter monitoring and control system may have more than one server (2). In the latter case, different system servers (2) can be connected to different ALVs (1), or all system servers (2) can be connected to all ALVs (1) in the system.

The communication (4) between the ALV (1) and the server (2) can be realized both by physical connections and by wireless communication. In one implementation option, the ALV (1) and the server (2) are connected to a common internal network of the medical institution by physical connections. In another implementation option, the ALV (1) is connected to the server (2) via a mobile Internet communication (e.g. 2G/3G, 4G or 5G), wireless communication (e.g. wi-fi, Bluetooth) or other wireless transmission technologies. When connected via a mobile Internet communication, the ALVs (1) may be physically distant from each other, e.g. ALV (1) can be located in different buildings of the treatment facility or in different cities. The monitoring and control system of ALV parameters allows to combine even very distant ALVs (1), so ALV parameters can be monitored and managed remotely by competent personnel of the medical institution, and ALV (1) can be serviced by a lower-qualified doctor. This is especially true in the case of a pandemic, with a shortage of doctors.

The ALV parameter monitoring and control device (3) is a computer, tablet, mobile phone or other smart device connected to the server (2) and capable of monitoring and controlling the ALV (1) parameters remotely. Therefore, the user can monitor and control the parameters of many ALVs (1) from the “clean zone” where the ALV parameter monitoring and control device (3) is located. There is no need to go to the patient's ward, which reduces the chance of infection and saves time, which can be critical to the health of patients. The ALV parameter monitoring and control device (3) includes the ALV parameter monitoring module (3.1) and the ALV parameter monitoring and control module (3.2).

The ALV parameter monitoring module (3.1) enables three-level data monitoring in the ALV parameter monitoring and control device (3). The first level data provides information about the ALV (1) parameter monitoring and control system itself. This can display: the amount of ALV (1) in the system, the location of ALV (1) in the treatment facility, the mode of ALV (1) in use, the charge level of the backup power battery (1.4), date and time, alarms, event history, two-way voice communication module (6) activation icon, menu icon, breathing gas data or other data.

The second level data provides the patient card and the parameters of the specific ALV (1). The patient card registration window is filled every time a new patient is connected to the ALV (1). As an example, the following patient information may be summarized: patient type, sex, height, weight, whether or not an intubation tube is used, if used, its diameter, which is required to calculate automatic air resistance compensation, etc.

Level 3 data is for the ALV (1) service. The service user can monitor wear parts status, system settings, event log, etc.

The ALV parameter monitoring module (3.1) remotely provides information about the ALV (1) parameter monitoring and control system itself, the patient card and all ALV (1) parameters that are visible directly to the ALV (1). Since changes in even one parameter of the ALV (1) can significantly affect the performance of artificial ventilation and the safety of the patient, all essential parameters of the ALV (1) must be represented in the device for monitoring and controlling the ALV parameters (3). Remotely displayed ALV (1) parameters can be: oxygen concentration (FiO2), respiratory rate (RR), inspiratory time (Tinsp), expiratory time (Texp), inspiratory pressure (Pinsp), inspiratory-expiratory time ratio (I:E ratio), inspiratory single volume (VTinsp), maximum pressure (Pmax), positive end-expiratory pressure (PEEP), determination of spontaneous breathing sensitivity (Spont trigger), selection of backup mode (Backup mode) and pressure maintenance (Pressure support), total ventilation time, inspiratory minute volume (MVinsp), expiratory single volume (VTexp), expiratory minute volume (MVexp), respiratory gas leakage (Air leak), total respiratory rate (Rtot), respiratory gas flow (Flow), lung tissue continuity (Compliance), peak pressure (Ppeak), pressure plateau indicator (Pplato), oxygen saturation (SpO2), end-expiratory carbon dioxide content (ETCO2), respiratory gas flow, pressure-volume curves, pulse oximetry and CO2 curves. The ALV parameter monitoring module (3.1) also remotely displays ventilation modes and alarms.

The parameters of ALV (1) can be represented by numerical expressions or graphs. The ALV parameters monitoring module (3.1) can display both static values of ALV (1) parameters and changes of ALV (1) parameters over time. The values of the ALV (1) parameters seen by the ALV (1) directly are duplicated in the ALV parameter monitoring and control device (3) in real time.

The ALV parameter monitoring and control module (3.2) allows you to remotely change selected ALV (1) parameters, lung ventilation modes and alarm settings. The ventilation mode and at least one of the following ALV (1) parameters can be changed remotely: oxygen concentration (FiO2), respiratory rate (RR), inspiratory time (Tinsp), expiratory time (Texp), inspiratory pressure (Pinsp), inspiratory-expiratory time ratio (I:E ratio), inspiratory unit volume (VTinsp), peak pressure (Pmax), positive end-expiratory pressure (PEEP), determination of spontaneous breathing sensitivity (Spont trigger), selection of backup mode (Backup mode) and pressure maintenance (Pressure support), total ventilation time, inspiratory minute volume (MVinsp), expiratory single volume (VTexp), expiratory minute volume (MVexp), respiratory gas leakage (Air leak), total respiratory frequency (Rtot), respiratory gas flow (Flow), lung tissue compliance (Compliance), peak pressure (Ppeak), pressure plateau index (Pplato), oxygen saturation (SpO2) and end-expiratory carbon dioxide content (ETCO2). When more than one ALV (1) parameter is changed, only one ALV (1) parameter is changed at a time, which can change another ALV (1) parameter. After changing one ALV (1) parameter remotely, a certain time interval is given to confirm or cancel the changes. In one implementation option, it is recommended to wait at least 3 min. during which the ALV (1) parameter monitoring and control system indicates that the change is valid, no alarms were generated after the change. The alarm settings of the selected ALV (1) or the alarm settings of any ALV (1) in the system can be changed remotely. When changing the ALV (1) parameters remotely, the control commands of the ALV parameters monitoring and control device (3) must reach the ALV (1) as soon as possible.

In one implementation option, the ALV parameter monitoring and control device (3) sends data and control commands to the server (2) and receives from the server (2) using a web browser. IP+TCP+MQTT data transfer model can be used for data transfer. HTTP, HTTPS, FTP, SMTP or other protocols may be used on a case-by-case basis. In a common case, in medical institutions, in order to save a computer program on a computer, it is necessary to contact the IT department. In this case, all ALVs (1) in the system can be easily and remotely accessed without having to install a computer program.

In another implementation option, the ALV parameter monitoring and control device (3) sends to the server (2) and receives data and control commands from the server (2) with the help of a program recorded in the ALV parameter monitoring and control device (3). In this case, ALV (1) parameters have greater monitoring and control functionality.

The ALV (1) parameter monitoring and control system may have more than one ALV parameter monitoring and control device (3). For example, a doctor can monitor ALV (1) remotely from a stationary, main device for monitoring and controlling ALV parameters (3). In a separate case, the ALV parameter monitoring and control device (3) can be a mobile phone or other mobile device.

The server (2) and the ALV parameter monitoring and control device (3) can be connected by physical connections, wireless communication (e.g. wi-fi, Bluetooth communication) or mobile Internet communication (e.g. 2G/3G, 4G or 5G), or other wireless transmission technologies. The communication (5) between the server (2) and the ALV parameter monitoring and control device (3) is duplicated. In an emergency, when communication (5) becomes unstable or disappears altogether, communication (5) is automatically replaced by a backup communication (5). For example, when the Wi-Fi wireless communication (5) between the server (2) and the monitoring and control device (3) is lost, the communication (5) is automatically replaced by the internal network communication of the medical facility with physical connections or a mobile Internet communication.

In one implementation option, the maximum delay in transmitting signals between the ALV (1) and the remote ALV parameters monitoring and control device (3) is no more than 3 s.

The communication (5) between the server (2) and the monitoring and control device (3) is encrypted. SSL/TLS/HTTPS protocols and SSL certificates, AES-256 encryption, or other data security and encryption protocols can be used for data transmission. During data transmission, they are inaccessible to outsiders, so the monitoring and control system of ALV parameters is safe and dependable.

The communication (5) between the server (2) and the monitoring and control device (3) requires authorization and identification. The user of the system must create his own account, and in order to log in, he must enter a username, password or other means of identification, so outsiders cannot remotely monitor or change the ALV (1) parameters.

The ALV parameter monitoring and control device (3) has a two-way voice communication module (6) that provides voice communication between the ALV (1) and the ALV parameter monitoring and control device (3). The two-way voice communication module (6) uses data transmission channels different from those used by the ALV (1) to transmit data between the ALV (1) and the ALV parameter monitoring and control device (3). In a separate case, the two-way voice communication module (6) is implemented via a mobile Internet communication. The two-way voice communication module (6) includes an interactive data monitoring and transmission device.

In one implementation option, the two-way voice communication module (6) is integrated into the ALV parameter monitoring and control device (3) and each ALV (1). Both the ALV (1) and the ALV parameter monitoring and control device (3) have a two-way voice communication module (6) icon, which, when pressed, generates a two-way voice communication between the ALV (1) and the ALV parameter monitoring and control device (3). The two-way voice communication module (6) additionally has visual means to inform with which ALV (1) a two-way voice communication is established. The system user located at the ALV (1) and the patient can communicate directly with other doctors located at the ALV parameter monitoring and control device (3), so a real-time remote consultation or consultation can be requested. In this way, a doctor with less competence can be present at the ALV (1) and the patient and work remotely under the supervision of a more experienced doctor. Only one audio call is possible with the ALV parameter monitoring and control device (3) at a time.

In another implementation option, the two-way voice communication module (6) is one or more stand-alone mobile stations not integrated into the ALV (1). In this case, the user carries the two-way voice communication module (6) station with him when visiting patients. The mobile station may have a wireless headset that transmits data between the ALV (1) and the mobile station. The data is transmitted through different channels than the channels for data transmission between the ALV (1) and the ALV parameter monitoring and control device (3). The advantage of the mobile station is that the two-way voice communication signal is free from extraneous interference from breathing gas pumps, ventilators, other device signals or patient noise.

In another implementation option, the two-way voice communication module (6) includes a headset and a microphone with direct uninterrupted communication with remote audio devices. In this case, the doctor has a headset and a microphone, and these devices maintain continuous communication with the remote audio facilities located in the ALV (1). Therefore, the doctor can maintain voice communication with the ALV parameter monitoring and control device (3) not only when he is close to the ALV (1), but also at a distance from the ALV (1).

In a preferred implementation option of the described invention, the two-way voice communication module (6) is integrated into the ALV (1), and the voice communication between the ALV (1) and the ALV parameter monitoring and control device (3) is transmitted through the server (2). In another implementation option, the two-way voice communication module (6) is also integrated into the ALV (1), and the voice communication between the ALV parameter monitoring and control device (3) and the ALV (1) is direct without using a server (2).

In one implementation option, the server (2) is additionally connected to the alarm system (7) of the medical facility. Integration with the alarm system is realized through open collector outputs or relays. The server (2) can be connected with the alarm system (7) by wired connection according to the HL7 standard. If the ALV (1) parameters exceed the limit values, the server (2) automatically sends an alarm signal to the alarm system (7). ALV (1) and ALV parameter monitoring and control device (3). The alarm system (7) generates messages of various levels according to the importance of the patient's condition, these messages of various levels are presented by different audio and/or visual signals. When the alarm system (7) has more than one alarm station (7.1), the alarm signal from the server (2) is sent to the alarm station (7.1) nearest to the patient. If the alarm does not disappear, it is sent to further alarm stations (7.1). If the alarm still persists, it is sent to further alarm stations (7.1). The alarm signal is forwarded to other alarm stations (7.1) after a certain period of time, which may affect the patient's health or life, depending on the importance of the ALV (1) parameters, the patient's condition, age or other parameters.

In another implementation option, there is no central alarm system (7) in the ALV (1) parameter monitoring and control system. In this case, if the ALV (1) parameters exceed the limit values, the server (2) automatically sends an alarm signal to the ALV (1). Since the ALV parameter monitoring and control device (3) reproduces the images seen on the ALV (1), the ALV parameter monitoring and control device (3) will also display alarms. The user can change the parameters of ALV (1) both directly at the patient and remotely. After changing the required parameters of the ALV (1), the alarm on both the ALV (1) and the ALV parameter monitoring and control device (3) disappears. Therefore, the user can monitor and change the parameters of the ALV (1) and monitor patient changes both directly on the ALV (1) and remotely via the ALV parameter monitoring and control device (3).

The ALV (1) parameter monitoring and control method has the following steps:
data transfer between ALV (1) and server (2);
data transfer between the server (2) and the ALV parameters monitoring and control device (3);
remote monitoring of ventilation modes, alarms and all ALV (1) parameters from the ALV parameters monitoring and control device (3);
changing ventilation modes and at least one ALV (1) parameter remotely from the ALV parameter monitoring and control device (3).

### Data Transfer Between ALV (1) and Server (2)

In one implementation option, the ALV (1) parameter monitoring and control system comprises more than one ALV (1), a server (2) and one ALV parameter monitoring and control device (3). When the ALV (1) and the server (2) are connected to the common internal network of the medical institution by physical connections, the data is transferred by physical connections. In another implementation option, the data between the ALV (1) and the server (2) is transmitted via a mobile Internet communication (e.g. 2G/3G, 4G or 5G), wireless communication (e.g. wi-fi. Bluetooth) or other wireless transmission technologies. Data transmission is redundant: in the event of failure or interruption of the main data transmission communication, the backup data transmission communication is automatically activated.

### Data Transfer Between the Server (2) and the ALV Parameter Monitoring and Control Device (3)

Data between the server (2) and the ALV parameter monitoring and control device (3) can be transferred either by physical connections or by wireless communication (e.g. wi-fi. Bluetooth), or mobile Internet communication (e.g. 2G/3G, 4G, 5G) or other wireless transmission technologies. The data being sent is encrypted, so the system is protected against unauthorized access to the system during data transmission. Additionally, the communication (5) between the ALV parameter monitoring and control device (3) and the server (2) requires authorization and identification. Only a registered user can remotely connect to the system—outsiders, without a login account and passwords, cannot connect to the ALV (1) parameter monitoring and control system. Data transmission is redundant: in the event of failure or interruption of the main data transmission communication, the backup data transmission communication is automatically activated.

Remote Monitoring of Ventilation Modes, Alarms and all ALV (1) Parameters from the ALV Parameter Monitoring and Control Device (3)

The ALV parameter monitoring and control device (3) remotely displays information about the ALV (1) parameter monitoring and control system itself, ventilation modes, patient card, alarms and all ALV (1) parameters that are visible directly on the ALV (1). The ALV parameter monitoring and control device (3) receives data from the server (2) and displays it on the screen. Three levels of data are displayed. The first level data provides data on the monitoring and control system of the ALV (1) parameters itself: the amount of ALV (1) in the system, the location of the ALV (1) in the treatment facility, the ALV (1) mode used, the charge level of the backup power battery (1.4), the date and time, alarms, event history, two-way voice module (6) activation icon, menu icon, breath gas data, etc. The second level data provides information about the patient card, the parameters of the selected ALV (1) and the change of parameters over time. The user can view the desired ALV (1) parameters remotely. Level 3 data is for ALV (1) service: automatic settings can be changed, software can be updated and other changes can be made remotely. Data between the ALV (1) and the ALV parameter monitoring and control device (3) are displayed in real time.

In one implementation option, the ALV (1) parameter monitoring and control system includes an ALV (1), a server (2) and an ALV parameter monitoring and control device (3). The server (2) forwards data between the ALV (1) and the ALV parameter monitoring and control device (3), so it is possible to remotely monitor the parameters of the ALV (1) and other related information about the ALV (1) in real time.

In another implementation option, the ALV (1) is directly connected to the ALV (1) parameter monitoring and control device (3) without using a server (2). In this case, the ALV (1) has an Internet service (1.5), which provides direct communication between the ALV (1) and the ALV (1) parameter monitoring and control device (3). In this case, the user receives the ALV (1) parameter data directly from the ALV (1).

Changing Ventilation Modes and at Least One ALV (1) Parameter Remotely from the ALV Parameter Monitoring and Control Device (3)

In one implementation option, the ALV (1) parameter monitoring and control system includes an ALV (1), a server (2) and an ALV parameter monitoring and control device (3). To change the ALV (1) parameter values, the user enters the desired ALV (1) parameter values into the ALV (1) parameter monitoring and control device (3), which sends control commands to the server (2), and the server (2) forwards the control commands to ALV (1). In this way, ALV (1) parameters or ventilation mode can be changed remotely. It is possible to change the parameters of more than one ALV (1). As an example, a system can have 20 ALVs (1), so that the parameters of 20 ALVs (1) can be changed remotely.

In another implementation option, when the ALV (1) is directly connected to the ALV (1) parameter monitoring and control device (3), without using a server (2), the ALV (1) has an Internet service (1.5). In this case, the user can change the data of the ALV (1) parameters in direct communication between the ALV (1) and the ALV (1) parameter monitoring and control device (3) without using the server (2). The user sends control commands from the ALV (1) to the ALV parameter monitoring and control device (3).

The ALV parameter monitoring and control device (3) has two-way voice communication (6) with the ALV (1), so the user at the ALV (1) can consult with colleagues at the ALV (1) parameter monitoring and control device (3).

In order to illustrate and describe this invention, the above description of the most appropriate implementation options is of a general nature—the description presents the measurements of the parts that make up the ALV (1) parameter monitoring and control system, the materials, the method of communication, the amount of the parts themselves and other parameters, and the method of use and purpose of the devices can differ—the description should be viewed as an illustration and not as a limitation. This is not an exhaustive or limiting description intended to determine a precise form or implementation option. Modifications may be made to implementation options described by those skilled in the art without departing from the scope of the present invention as defined below.

### CLAUSES

Clause 1. Monitoring and control system of artificial lung ventilation device (ALV) parameters, comprising an ALV (1), server (2) and monitoring and control device (3),
wherein:
   the system includes more than one ALV (1);
   each ALV (1) has a patient identification device (1.3),
   the server (2) has a layout scheme of ALVs in the premises (2.4) and has a communication with a ALV parameter monitoring and control device (3) and more than one ALV (1);
   the ALV parameter monitoring and control device (3) is remote, has a ALV parameter monitoring module (3.1) and a ALV parameter monitoring and control module (3.2);
   the system has two-way voice communication (6) between the ALV (1) and the ALV parameter monitoring and control device (3);
therefore, the user can remotely monitor and change the ventilation mode, ALV parameters or alarms of more than one ALV (1) using the ALV parameter monitoring and control device (3).

Clause 2. The ALV parameter monitoring and control system according to clause 1, wherein the communication (4, 5) between the ALV (1), the server (2) and the ALV parameter monitoring and control device (3) is optionally one of the following: an internal network with physical connections, wireless communication (wi-fi, Bluetooth), mobile Internet communication (2G/3G/4G or 5G) or other wireless technologies.

Clause 3. ALV parameter monitoring and control system according to clause 1-2, wherein the Internet of Things unit (1.1) is either integrated into the ALV (1) or is available as a stand-alone attachment, connected to the ALV (1) by external connectors;
when the IoT unit (1.1) is a stand-alone attachment, it is easily replaceable, so one wireless communication technology can be easily replaced by another wireless communication technology in the system.

Clause 4. ALV parameter monitoring and control system according to clause 1-3, wherein the ALV (1) has an Internet service (1.5), in this case, the ALV (1) is connected directly to the ALV parameter monitoring and control device (3) without using a server (2).

Clause 5. ALV parameter monitoring and control system according to clause 1-4, wherein the ALV (1) parameter monitoring and control system has a two-way voice communication module (6); the two-way voice communication module (6) is integrated into the ALV parameter monitoring and control device (3) and each ALV (1): or the two-way voice communication module (6) is one or more stand-alone mobile stations not integrated into the ALV (1); or the two-way voice communication module (6) is a headset and microphone with direct uninterrupted communication with remote audio devices located in the ALV (1).

Clause 6. ALV parameter monitoring and control system according to clause 5, wherein the two-way voice communication module (6) uses data transmission channels different from those used by the ALV (1) to transmit data between the ALV (1) and the ALV parameters monitoring and control device (3).

Clause 7. ALV parameter monitoring and control system according to clause 1-6, wherein the ALV parameter monitoring and control device (3) sends to the server (2) and receives data and control commands from the server (2) using a web browser or to the ALV parameter monitoring and control device (3) with the help of a installed computer program.

Clause 8. ALV parameter monitoring and control system according to clause 1-7, wherein the communication (5) between the server (2) and the ALV parameter monitoring and control device (3) is encrypted.

Clause 9. ALV parameter monitoring and control system according to clause 1-8, wherein the communication (5) between the server (2) and the ALV parameter monitoring and control device (3) requires authorization and identification.

Clause 10. ALV parameter monitoring and control system according to clause 1-9, wherein the communication (5) between the server (2) and the ALV parameter monitoring and control device (3) is duplicated.

Clause 11. ALV parameter monitoring and control system according to clause 1-10, wherein the ALV parameter monitoring module (3.1) remotely and in real time provides information about the ALV (1) parameter monitoring and control system itself, the patient card, the used ventilation mode and all ALV (1) parameters that are visible directly in ALV (1).

Clause 12. ALV parameter monitoring and control system according to clause 11, wherein the ALV parameter monitoring module (3.1), ALV parameter monitoring and control device (3) provides three levels of data: the first level data provides information about the ALV parameter monitoring and control system itself, the second level data provides information about the patient card, the parameters of the specific ALV (1) and the ventilation mode, and the third level data is for the ALV (1) service.

Clause 13. ALV parameter monitoring and control system according to clause 1-12, wherein the ALV parameter monitoring and control module (3.2) can remotely change ventilation modes and at least one of the following ALV (1) parameters: oxygen concentration (FiO2), respiratory rate (RR), inspiratory time (Tinsp), expiratory time (Texp), inspiratory pressure (Pinsp), inspiratory-expiratory time ratio (I:E ratio), inspiratory unit volume (VTinsp), maximum pressure (Pmax), positive end-expiratory pressure (PEEP), determination of spontaneous breathing sensitivity (Spont trigger), selection of backup mode (Backup mode) and pressure maintenance (Pressure support), total ventilation time, inspiratory minute volume (MVinsp), expiratory single volume (VTexp), expiratory minute volume (MVexp), respiratory gas leakage (Air leak), total respiratory rate (Rtot), respiratory gas flow (Flow), lung tissue compliance (Compliance), peak pressure (Ppeak), pressure plateau index (Pplato), oxygen saturation (SpO2) or end-expiratory carbon dioxide (ETCO2).

Clause 14. ALV parameter monitoring and control system according to clause 1-13, wherein the ALV parameter monitoring and control system has an integrated alarm system (7) adapted for remote monitoring of ALV parameters, and if the ALV (1) parameters exceed the limit values, alarms are generated;

when the medical facility does not have an alarm system (7), the server (2) sends the alarms directly to the ALV (1).

Clause 15. A method for remotely monitoring and controlling ALV (1) parameters, ventilation modes, and alarms, comprising the following steps:
data transfer between ALV (1) and server (2);
data transfer between the server (2) and the ALV parameters monitoring and control device (3);
remote monitoring of ventilation modes, alarms and all ALV (1) parameters from the ALV parameter monitoring and control device (3);
remote change of ventilation modes and at least one ALV (1) parameter from the ALV parameter monitoring and control device (3).

What is claimed is:

1. A monitoring and control system of artificial lung ventilation device (ALV) parameters, comprising an ALV (1), server (2) and monitoring and control device (3), wherein:
the system includes more than one ALV (1);
each ALV (1) has a patient identification device (1.3),
the server (2) has a layout scheme of ALVs in the premises (2.4) and has a communication with an ALV parameter monitoring and control device (3) and more than one ALV (1);
the ALV parameter monitoring and control device (3) is remote, has a ALV parameter monitoring module (3.1) and a ALV parameter monitoring and control module (3.2);
the system has two-way voice communication (6) between the ALV (1) and the ALV parameter monitoring and control device (3);
wherein the user can remotely monitor and change the ventilation mode, ALV parameters or alarms of more than one ALV (1) using the ALV parameter monitoring and control device (3).

2. The monitoring and control system according to claim 1, wherein the communication (4, 5) between the ALV (1), the server (2) and the ALV parameter monitoring and control device (3) is optionally one of the following: an internal network with physical connections, wireless communication (wi-fi, Bluetooth), mobile Internet communication (2G/3G/4G or 5G) or other wireless technologies.

3. The monitoring and control system according to claim 1, wherein the Internet of Things unit (1.1) is either integrated into the ALV (1) or is available as a stand-alone attachment, connected to the ALV (1) by external connectors;
when the IoT unit (1.1) is a stand-alone attachment, it is easily replaceable, so one wireless communication technology can be easily replaced by another wireless communication technology in the system.

4. The monitoring and control system according to claim 1, wherein the ALV (1) has an Internet service (1.5), in this case, the ALV (1) is connected directly to the ALV parameter monitoring and control device (3) without using a server (2).

5. The monitoring and control system according to claim 1, wherein the ALV (1) parameter monitoring and control system has a two-way voice communication module (6); the two-way voice communication module (6) is integrated into the ALV parameter monitoring and control device (3) and each ALV (1); or the two-way voice communication module (6) is one or more stand-alone mobile stations not integrated into the ALV (1); or the two-way voice communication module (6) is a headset and microphone with direct uninterrupted communication with remote audio devices located in the ALV (1).

6. The monitoring and control system according to claim 5, wherein the two-way voice communication module (6) uses data transmission channels different from those used by the ALV (1) to transmit data between the ALV (1) and the ALV parameters monitoring and control device (3).

7. The monitoring and control system according to claim 1, wherein the ALV parameter monitoring and control device (3) sends to the server (2) and receives data and control commands from the server (2) using a web browser or to the ALV parameter monitoring and control device (3) with the help of an installed computer program.

8. The monitoring and control system according to claim 1, wherein the communication (5) between the server (2) and the ALV parameter monitoring and control device (3) is encrypted.

9. The monitoring and control system according to claim 1, wherein the communication (5) between the server (2) and the ALV parameter monitoring and control device (3) requires authorization and identification.

10. The monitoring and control system according to claim 1, wherein the communication (5) between the server (2) and the ALV parameter monitoring and control device (3) is duplicated.

11. The monitoring and control system according to claim 1, wherein the ALV parameter monitoring module (3.1) remotely and in real time provides information about the ALV (1) parameter monitoring and control system itself, the patient card, the used ventilation mode and all ALV (1) parameters that are visible directly in ALV (1).

12. The monitoring and control system according to claim 11, wherein the ALV parameter monitoring module (3.1), ALV parameter monitoring and control device (3) provides three levels of data: the first level data provides information about the ALV parameter monitoring and control system itself, the second level data provides information about the patient card, the parameters of the specific ALV (1) and the ventilation mode, and the third level data is for the ALV (1) service.

13. The monitoring and control system according to claim 1, wherein the ALV parameter monitoring and control module (3.2) can remotely change ventilation modes and at least one of the following ALV (1) parameters: oxygen concentration (FiO2), respiratory rate (RR), inspiratory time (Tinsp), expiratory time (Texp), inspiratory pressure (Pinsp), inspiratory-expiratory time ratio (I:E ratio), inspiratory unit volume (VTinsp), maximum pressure (Pmax), positive end-expiratory pressure (PEEP), determination of spontaneous breathing sensitivity (Spont trigger), selection of backup mode (Backup mode) and pressure maintenance (Pressure support), total ventilation time, inspiratory minute volume (MVinsp), expiratory single volume (VTexp), expiratory minute volume (MVexp), respiratory gas leakage (Air leak), total respiratory rate (Rtot), respiratory gas flow (Flow), lung tissue compliance (Compliance), peak pressure (Ppeak), pressure plateau index (Pplato), oxygen saturation (SpO2) or end-expiratory carbon dioxide (ETCO2).

14. The monitoring and control system according to claim 1, wherein the ALV parameter monitoring and control system has an integrated alarm system (7) adapted for remote monitoring of ALV parameters, and if the ALV (1) parameters exceed the limit values, alarms are generated;
wherein when the medical facility does not have an alarm system (7), the server (2) sends the alarms directly to the ALV (1).

15. A method for remotely monitoring and controlling ALV (1) parameters, ventilation modes, and alarms, comprising the following steps:
data transfer between ALV (1) and server (2);
data transfer between the server (2) and the ALV parameters monitoring and control device (3);
remote monitoring of ventilation modes, alarms and all ALV (1) parameters from the ALV parameter monitoring and control device (3);
remote change of ventilation modes and at least one ALV (1) parameter from the ALV parameter monitoring and control device (3).
